(19) United States
(12) Patent Application Publication
(10) Pub. No.: US 2026/0165742 A1
(43) Pub. Date: Jun. 18, 2026

(54) NON-CLINICAL CONSTRUCT OPTIMIZER FOR HEXAPOD

(71) Applicant: DePuy Synthes Products, Inc., Raynham, MA (US)

(72) Inventors: Scott Lavoritano, West Chester, PA (US); Andreas Gamber, Raynham, MA (US); Horst Steigner, Raynham, MA (US); Bernd Gutmann, Raynham, MA (US); Albert Montello, West Chester, PA (US); Michael Wahl, Raynham, MA (US); Talissa Pryor, Raynham, MA (US)

(73) Assignee: DePuy Synthes Products, Inc., Raynham, MA (US)

(21) Appl. No.: 18/980,755

(22) Filed: Dec. 13, 2024

(51) Int. Cl. A61B 17/62

(52) U.S. Cl. CPC A61B 17/62

(57) ABSTRACT

An external fixator system may be used for a procedure on a patient. The external fixator system may include a hexapod, which for example may include a plurality of components. Example components may include one or more rings and one or more struts. For example, the hexapod may include an upper ring, a lower ring, and a strut. The strut may be included in a plurality of struts, for example of various sizes. A system may include a computer-readable medium used to determine a subset of components. The computer-readable medium may be stored, for example in a memory and may include instruction stored thereon instructions. The instructions may be executed by a processor. When executed, the system may determine the subset of components. The system may determine the subset of components based on one or more of the constraints and/or the range of possible values for the constraint.

### BACKGROUND

A bone deformity correction system may utilize a fixator, for example a hexapod, external to the patient. The hexapod may be utilized to correct the bone deformity, for example before or after a surgical procedure. The hexapod may be affixed to the patient near the bone deformity such that bone growth is controlled by the hexapod.

Positioning of the hexapod may be based on imaging. For example, images of the bone deformity may be input into a system with a graphical representation of the bone deformity and the hexapod. Positioning of the hexapod may then be determined such that bone growth is controlled as desired. However, one or more struts of the hexapod may need to be swapped as an initial position of the hexapod may be different from a final position of the hexapod such that one or more different struts are necessary at the final position. Strut swaps require an additional clinical visit for the patient, as well as medical professional and hexapod service representative efforts. This leads to additional expenses and potential complications, for example including possible injury to the patient during a strut swap.

### SUMMARY

The application is generally related to non-clinical devices and methods used determining hexapod struts, for example in a medical context, and more particularly, to minimizing strut swaps. In some examples, the devices and methods described herein can be used to indicate to a user, for example a surgeon, a subset of components (e.g., rings and/or struts) for use in a medical procedure.

An external fixator system may be used for a procedure on a patient. The external fixator system may include a hexapod, which for example may include a plurality of components. Example components may include one or more rings and one or more struts. For example, the hexapod may include an upper ring, a lower ring, and a strut. The strut may be included in a plurality of struts, for example of various sizes. A system may include a computer-readable medium used to determine a subset of components. The computer-readable medium may be stored, for example in a memory and may include instruction stored thereon instructions. The instructions may be executed by one or more processors. When executed, the system may determine the subset of components. The system may determine the subset of components based on one or more of the constraints and/or the range of possible values for the constraint.

The system may receive the one or more constraints, for example from user input (e.g., on a user interface). Example constraints include a tolerance in the upper ring location, a tolerance in the lower ring location, a strut length, and/or a skin circumference. The strut length value may indicate a length of a strut, for example when configured between the upper ring mounting location and the lower ring mounting location. The range of values may indicate a tolerance in the lower ring mounting location, for example as a lower ring tolerance range. The upper range value may indicate a tolerance in the length of a strut, for example at an initial position of the strut.

The system may determine the subset of components based on minimizing strut swaps during patient treatment with the external fixator system. The system may determine a plurality of subsets of components. For example, the system may determine a first subset of components based on minimizing strut swaps. The system may determine a second subset of components based on the weight of the hexapod, for example a minimum weight. A display may be included in the system. The display may display an indication of the subset of components. For example, the display may display an indication of the first subset of components and the second subset of components, such that the user may select the first subset of components or the second subset of components. Additionally, or alternatively, the system may receive a second plurality of constraints (e.g., from user input), for example with one or more changes to the plurality of constraints. The system may determine, based on the second plurality of constraints, the subset (e.g., second subset) of components.

The system may display an indication of the range, for example associated with a ring. The range may include a volume within which the ring may be located. The user may input the range and/or the system may determine the range, for example based on the input range. The system may display the indication of the range as a graphical output of a ring. Additionally, or alternatively, the system may display the indication of the range as a numerical value. The system may display an arrow, for example indicating a direction of a strut and/or a ring during patient treatment with the external fixator system. For example, the arrow may indicate which direction the strut and/or ring will move during treatment.

The system may receive a value associated with one or more constraints (e.g., from user input). The system may (e.g., then) determine a component of the subset of components based on the one or more constraints. For example, the system may receive a value associated with the skin circumference and determine a ring of the subset of components. The ring may include an inner circumference greater than or equal to the value associated with the skin circumference, for example plus a predetermined offset. The predetermined offset may correspond to a value set by the user or set by the system. The predetermined off set may correspond to a value for a user to place two fingers, for example to mount a component.

The upper ring may include an upper ring inner circumference and/or the lower ring may include a lower ring inner circumference. The instructions may determine an range of the upper ring mounting location and/or the lower ring mounting location. The range may include a distance between a bone fragment and the upper ring inner circumference and/or the lower ring inner circumference. The range may be an anteroposterior (AP) range, a lateral (LAT) range, a medial (MED) range, an anterior range, or a proximal range.

The system may determine a distance between the upper ring and the lower ring, for example between a first location of the upper ring and a first location of the lower ring. The system may determine another distance between the upper ring and the lower ring, for example between a second location of the upper ring and a second location of the lower ring. The second location of the upper ring may be opposite to the first location of the upper ring, for example on an opposite side of a circumference of the upper ring. The second location of the lower ring may be opposite to the first location of the lower ring, for example on an opposite side of a circumference of the lower ring. The system may determine the upper ring mounting location and the lower ring mounting location based on the first distance and the second distance. The system may determine the upper ring mounting location and the lower ring mounting location based on the difference between the first distance and the second distance, for example being less than a threshold value.

The plurality of struts may be associated with different ranges of lengths. The system may provide an indication of the determined subset of components, for example for use during an operation. The indication may be displayed on a graphical user interface (e.g., display). The indication of the subset of components may include one or more color indicators. For example, a respective color indicator may correspond to each respective strut in the subset of components. Each strut may include a first end and a second end. The first end may be configured to connect to the upper ring, for example at an upper ring aperture. The second end may be configured to connect to the lower ring, for example at a lower ring aperture. The system may determine a position of the first end. For example, the position of the first end may be a first aperture of the upper ring. The system may determine a position of the second end. For example, the position of the second end may be a first aperture of the lower ring. The system may determine whether to include a strut in the subset of components based on the first position and the second position.

The system may determine whether to include a strut of the plurality of struts in the subset of components, for example based on the strut length value. Additionally, or alternatively, the system may determine a minimum number of struts of the subset of components. For example, the minimum number of struts may be for an entire procedure/operation. The minimum number of struts may minimize or eliminate the need for strut swaps during a procedure. The system may determine the minimum number of struts based on one or more of the strut length value, the lower range value, or the upper range value.

### BRIEF DESCRIPTION OF THE DRAWINGS

Many aspects of the disclosure can be better understood with reference to the following drawings. The components in the drawings are not necessarily to scale, with emphasis being placed upon clearly illustrating the principles of the present disclosure. Moreover, in the drawings, like reference numerals designate corresponding parts throughout the several views. Furthermore, each drawing contained in this provisional application includes at least a brief description thereon and associated text labels further describing associated details.

FIG. 1A is a view of an example of a hexapod.

FIG. 1B is a view of another example of a hexapod with strut connection locations.

FIG. 2 is a block diagram that illustrates an example of a computing device.

FIG. 3A is a view of an example interface for selection of ring constraints.

FIG. 3B is a view of an example interface for selection of deformity constraints.

FIG. 3C is a view of another example interface for selection of ring constraints.

FIG. 4A is a view of an example interface for selection of ring mounting constraints.

FIG. 4B is a view of another example interface for selection of ring mounting constraints.

FIG. 4C is a view of another example interface for selection of ring mounting constraints.

FIG. 5A is a view of an example interface for calculating a hexapod strut configuration.

FIG. 5B is a view of another example interface for calculating a hexapod strut configuration.

FIG. 6 is a view of an example interface of a calculated output hexapod location.

### DETAILED DESCRIPTION

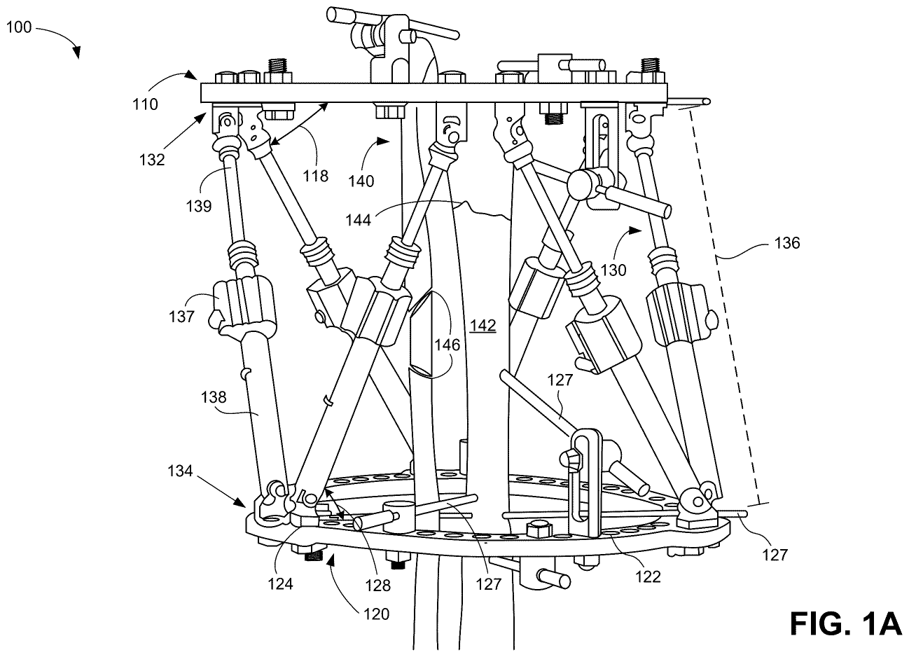

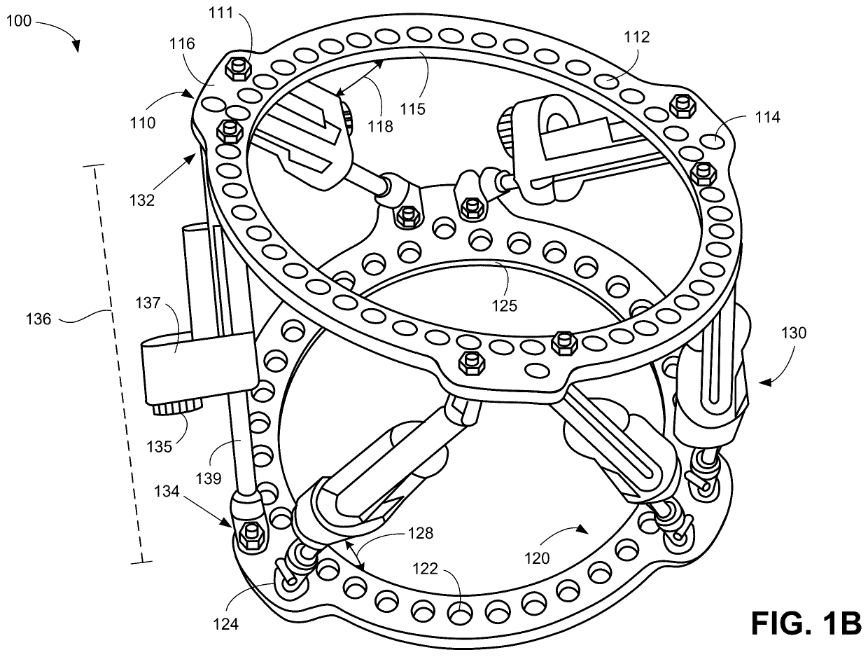

FIG. 1A is a view of an example of an external fixator system, such as a hexapod 100. The hexapod may include a first ring 110 and a second ring 120. The first ring 110 may be referred to as an upper ring, a proximal ring, or a reference ring. The second ring 120 may be referred to as a lower ring, a distal ring, or a moving ring. While FIG. 1A depicts the first ring 110 and second ring 120 as circular, the rings may be differently shaped, for example U-shaped, oblong, rectangular, triangular, or open.

The first ring 110 may be located near and/or coupled to a first bone fragment 140. The example first bone fragment 140 in FIG. 1A is depicted as a fibula, however, the first bone fragment 140 may be any bone. The second ring 120 may be located near and/or coupled to a second bone fragment 142. The example second bone fragment 142 in FIG. 1A is depicted as a tibia, however the second bone fragment 142 may be any bone. For example, the first bone fragment 140 and second bone fragment 142 may be on the same bone. The second bone fragment 142 may include a bone defect 144, for example a fracture. In order to support bone growth and correct the bone defect 144, a surgeon may perform an osteotomy, for example on the first bone fragment 140. The osteotomy may include one or more cuts 146 in the bone (e.g., the first bone fragment 140).

The first ring 110 and/or the second ring 120 may be coupled to the first bone fragment 140 and/or the second bone fragment 142, for example with a connector 127. Example connectors 127 include pins or wires. The connector 127 may keep the first ring 110 and/or the second ring 120 of the hexapod 100 in a fixed position with respect to the first bone fragment 140 and/or the second bone fragment 142. However, as the first bone fragment 140 grows, the position of the first ring 110 and/or the second ring 120 of the hexapod 100 may need to be adjusted to control the first bone fragment 140 growth. Adjustment of the length 136 of one or more of the struts 130 may be utilized to adjust the position of the first ring 110 and/or the second ring 120. Alternatively, or additionally, replacement of one or more struts 130 (e.g., strut swap) may be utilized to adjust the position of the first ring 110 and/or the second ring 120.

The first ring may include a plurality of first ring apertures (not shown), for example for mounting one or more struts 130. The second ring may include a plurality of second ring apertures 122, for example for mounting the one or more struts 130. The struts 130 may each include a first end 132 and a second end 134, for example such that the first end 132 is configured to couple to the one or more first ring apertures (not shown) and the second end is configured to couple to the one or more second ring apertures 122. Each strut 130 includes a length 136, which may be adjustable between a minimum length value and a maximum length value, for example for adjusting the position of the first ring 110 and/or the second ring 120.

The first end 132 and second end 134 of each strut 130 may be coupled to the first ring 110 and the second ring 120 respectively via a joint mechanism. For example, a joint mechanism may include a ball joint, a constrained hinge joint, or a universal joint. A joint mechanism may allow the strut 130 to have degrees of freedom with respect to the first ring 110 and/or the second ring 120. For example, a universal joint may allow the strut to have six degrees of freedom.

Initially a user (e.g., a surgeon) may couple the hexapod 110 to the patient, for example at the first bone fragment 140 and/or the second bone fragment 142. The hexapod 110 may be coupled at an initial position, for example as in FIG. 1A. However, as the first bone fragment 140 grows, adjustments may be made to the hexapod 100. For example, the position of the first ring 110 and/or the second ring 120 may change. A distance and/or an angle between the first ring 110 and the second ring 120 may be adjusted during a procedure. The procedure may involve the patient adjusting the length 136 of one or more struts 130 of the hexapod 100, for example at home.

In some examples the second ring 120 may be coupled to the first bone fragment 140 and/or the second bone fragment 142, and may remain fixed with respect to the bone fragments. The first ring 110 may move with respect to the first bone fragment 140 and/or the second bone fragment 142, for example when the length 136 of the one or more struts 130 is adjusted. The strut 130 may include a slider 136. The slider 136 may be moved (e.g., by a user) such that a sheath 138 of the strut 130 moves to at least partially cover a shaft 139 of the strut 130. As the sheath 138 covers the strut, the length 136 of the strut is decreased. The slider may additionally be moved (e.g., by a user) such that the sheath 138 moves to at least partially uncover the shaft 139 of the strut 130, such that as the sheath 138 uncovers the shaft 139 the length 136 of the strut is increased.

When a strut 130 reaches a minimum length value or a maximum length value and it is desirable to respectively shorten or lengthen the strut length 136 further, a strut swap may be desirable. As discussed, a strut swap may involve an additional clinical visit for the patient, as well as medical professional and hexapod service representative effort. For example, a first strut with a minimum length value of 60 mm and a maximum length value of 100 mm, may be utilized in the hexapod when the distance between the first ring 110 and the second ring 120 at the initial position is a first value (e.g., 80 mm). However, the distance between the first ring 110 and the second ring 120 at the final position may be a second value (e.g., 120 mm), such that the second value exceeds the maximum length value of the first strut. A strut swap may be needed for the first ring 110 and the second ring 120 to reach the final position.

Alternatively, or additionally, a user may move the second end 134 of the first hexapod strut 130 from a second ring first aperture 122 to a second ring second aperture 124. For example, the user may move the second end 134 of the first hexapod strut 130 from the second ring first aperture 122 to the second ring second aperture 124 on a protrusion portion 126 of the second ring 120. The user moving the second end 134 of the first hexapod strut 130 from the second ring first aperture 122 to the second ring second aperture 124 may change an angle 128 between the first strut 130 and the second ring 120 and/or the angle 118 between the first strut 130 and the first ring 210.

FIG. 1B is a view of another example of the hexapod 100 with strut connection locations. The first ring 110 may include first apertures 112. For example, first ring first apertures 112 may be distributed in a first row of ring apertures adjacent an inner surface 115 of the first ring 110. For example, the inner surface 115 of the first ring 110 may be proximate to the patient (e.g., the first bone fragment and/or second bone fragment). The first ring 110 may include first ring second apertures 114. For example, first ring second apertures 114 may be distributed in a second row of ring apertures opposite the inner surface 115 of the first ring 110. For example, first ring second apertures 114 may be on a protrusion portion 116 of the first ring 110.

Similarly, the second ring 120 may include first apertures 122. For example, second ring first apertures 122 may be distributed in a first row of ring apertures adjacent to an inner surface 125 of the second ring 120. For example, the inner surface 125 of the second ring 120 may be proximate to the patient (e.g., the first bone fragment and/or second bone fragment). The second ring 120 may include second ring second apertures 124. For example, second ring second apertures 124 may be distributed in a second row of ring apertures opposite the inner surface 125 of the second ring 120. For example, second ring second apertures 124 may be on a protrusion portion 126 of the second ring 120. The first end 132 and/or the second end 134 of a hexapod strut 130 may be coupled to the first ring 110 and/or the second ring 120, for example with a connection mechanism. For example, the connection mechanism may include threads (not shown) and/or a nut 111 configured to abut the first ring 110 and/or the second ring 120.

The strut 130 may include a slider 137 as discussed herein. Additionally, or alternatively, the strut 130 may include a knob 135. The knob 135 may be rotatably connected to the strut shaft 139 such that rotation of the knob increases or decreases the strut length 136. When a user uncouples a strut 130 from the first ring 110 and/or the second ring 120, the user may uncouple the connection mechanism from the first ring 110 and/or the second ring 120. The user may (e.g., then) recouple the strut 130 at a different aperture (e.g., 112, 114, 122, or 124). Alternatively, the user may remove the other connection mechanism from the other ring (e.g., first ring 110 or second ring 120) such that the strut is completely removed from the hexapod 100. The user may (e.g., then) swap the strut 130 (e.g., strut swap) for a different strut, for example with a desired strut length 136.

Devices may collect data from various data sources, for example from sensors or inputs. A device may determine one or more constraints as herein. The constraints may be associated with a patient and/or with an external fixator system. The device may include a computer-readable medium, for example used to determine the subset of components. The computer-readable medium may be stored, for example in a memory and may include instruction stored thereon instructions. The instructions may be executed by a processor. For example, the instructions may include one or more portions of the procedures described herein. Although described with reference to a hexapod, systems and methods described herein may include and/or be used with other external fixator systems.

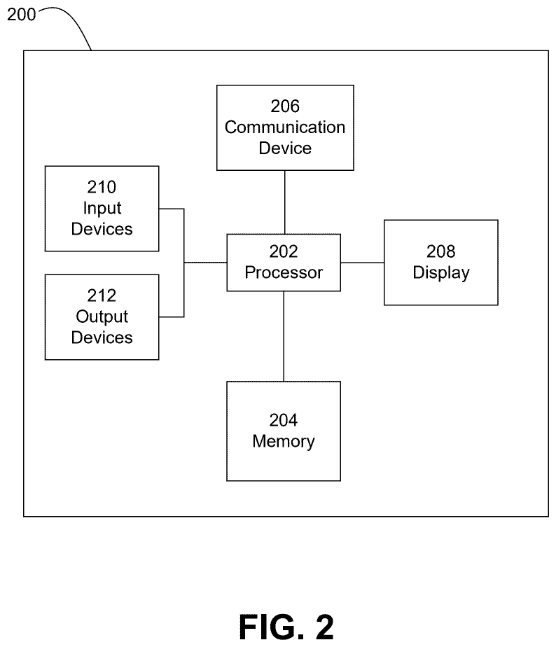

FIG. 2 is a simplified block diagram of an example device 200. The device 200 may be an example of a computing device (e.g., one or more servers), such as the computing device described herein. Alternatively, or additionally, the device 200 may be an example of a user interface described herein. In such instances, the device 200 may include a personal computer, such as a laptop or desktop computer, a tablet device, a cellular phone or smartphone, a server, or another type of client device. The device 200 may be configured to receive constraints, determine a subset of components, and/or display an indication of the subset of components as described herein. As shown by FIG. 2, the device 200 may comprise a processor 202, a memory 204, a communication device 206, a display 208, one or more input devices 210, and/or one or more output devices 212. It should be appreciated that the device 200 may include fewer or more components than those shown in FIG. 2.

The processor 202 may include one or more general purpose processors, special purpose processors, conventional processors, digital signal processors (DSPs), microprocessors, integrated circuits, a programmable logic device (PLD), application specific integrated circuits (ASICs), or the like. The processor 202 may perform signal coding, data processing, image processing, power control, input/output processing, and/or any other functionality that enables the device 200 to perform as described herein.

The processor 202 may store information in and/or retrieve information from the memory 204. The memory 204 may include a non-removable memory and/or a removable memory. The non-removable memory may include random-access memory (RAM), read-only memory (ROM), a hard disk, or any other type of non-removable memory storage. The removable memory may include a subscriber identity module (SIM) card, a memory stick, a memory card, or any other type of removable memory. The memory may be local memory or remote memory external to the device 200. The memory 204 may store instructions which are executable by the processor 202. Different information may be stored in different locations in the memory 204.

The memory 204 may comprise a computer-readable medium or machine-readable storage media that maintains computer-executable instructions for performing one or more as described herein. The computer-readable medium may be non-transitory. The memory 204 may comprise instructions, for example computer-executable instructions or machine-readable instructions, that include one or more portions of the procedures described herein. The processor 202 of the device 200 may access the instructions from memory for being executed to cause the processor 202 of the device 200 to operate as described herein. The memory 204 may comprise computer-executable instructions for executing configuration software. For example, the computer-executable instructions may be executed to perform, in part and/or in their entirety, one or more procedures as described herein. Further, the memory 204 may have stored thereon one or more settings and/or control parameters associated with the device 200.

The processor 202 may communicate with other devices via the communication device 206. The communication device 206 may transmit and/or receive information over a network, which may include one or more other devices. The communication device 206 may perform wireless and/or wired communications. The communication device 206 may include a receiver, transmitter, transceiver, or other device capable of performing wireless communications via an antenna. The communication device 206 may be capable of communicating via one or more protocols, such as a cellular communication protocol, a Wi-Fi communication protocol, Bluetooth®, a near field communication (NFC) protocol, an internet protocol, another proprietary protocol, or any other radio frequency (RF) or communications protocol. The device 200 may include one or more communication devices 206.

The processor 202 may be in communication with a display 208 for providing information to a user (e.g., surgeon). The information may be provided via an interface on the display 208. The information may be provided as an image generated on the display 208. The display 208 and the processor 202 may be in two-way communication, as the display 208 may include a touch-screen device capable of receiving information from a user (e.g., surgeon, patient, etc.) and providing such information to the processor 202. The processor 202 may be configured to generate, on the display 208, an indication of one or more notifications described herein, such as an indication of the progression of MSA of a patient, etc. The processor 202 may be configured to generate, on the display 208, an indication of one or more notifications described herein, such as the indication of the subset of hexapods. The processor 202 may be configured to generate an algorithm, for example based on the constraints.

The processor 202 may be in communication with input devices 210 and/or output devices 212. The input devices 210 may include a camera, a microphone, a keyboard or other buttons or keys, a mouse, and/or other types of input devices for sending information to the processor 202. The display 208 may be a type of input device, as the display 208 may include touch-screen sensor capable of sending information to the processor 202. The output devices 212 may include speakers, indicator lights, or other output devices capable of receiving signals from the processor 202 and providing output from the device 200. The display 208 may be a type of output device, as the display 208 may provide images or other visual display of information received from the processor 202.

Although not illustrated, the device 200 may include a power supply. In some examples, the power supply may include one or more batteries. In some examples, the power supply may include an AC to DC power converter. The power supply may be configured to power the processor 202 and the other low voltage circuitry of the device 200.

Although not illustrated, the device 200 may include a GPS circuit (e.g., in instances where the device 200 is a client device). The processor 202 may be in communication with the GPS circuit for receiving geospatial information. The processor 202 may be capable of determining the GPS coordinates of the device 200 based on the geospatial information received from the GPS circuit. The geospatial information may be communicated to one or more other communication devices to identify the location of the device 200.

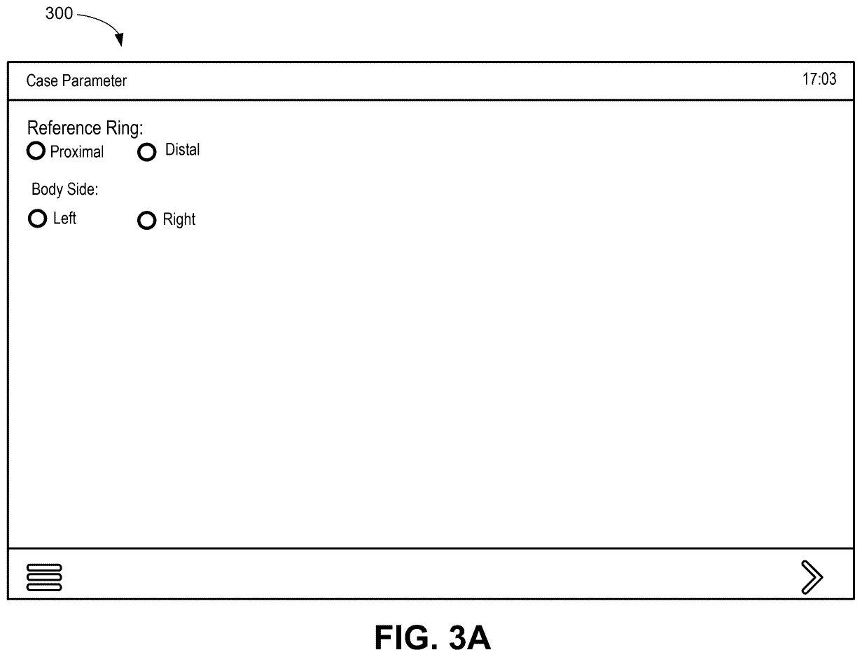

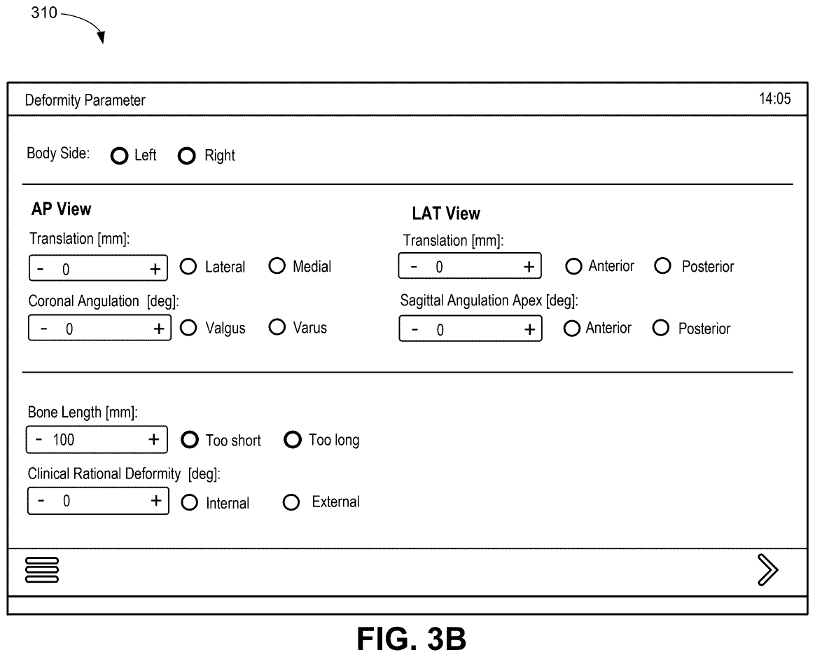

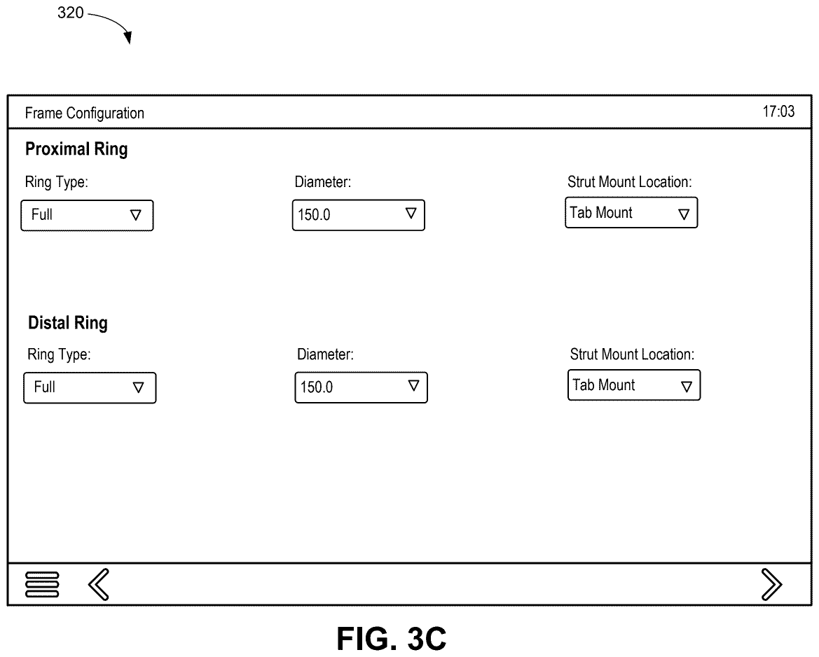

FIG. 3A is a view of an example interface 300 for selection of ring constraints. A user may select a ring, for example a proximal ring (e.g., first ring 110) or a distal ring (e.g., second ring 120) should be the reference ring. Additionally, or alternatively, the user may select whether the ring (e.g., proximal ring or distal ring) is to be mounted on the left or right body side. The user may select the ring (e.g., proximal ring or distal ring) based on deformity constraints. Example deformity constraints may include a bone length (e.g., of each fragment), a clinical rational deformity, a translation, a vertical translation, a coronal angulation, a sagittal angulation apex, a body side (e.g., left or right), a strain (e.g., at the fracture), and/or a stiffness (e.g., of the fracture).

FIG. 3B is a view of an example interface 310 for selection of deformity constraints. Deformity constraints may be based on observation of a bone deformity, for example from imaging. Deformity constraints may include a bone length, for example for each bone fragment. Example bone fragments may include the first bone fragment 140 and/or the second bone fragment 142. The user may enter the body side (e.g., left or right) where the deformity is located. The interface 310 may include a constraint range for (e.g., each of) the constraints. For example the constraints may include one or more of an anteroposterior (AP) constraint, a lateral (LAT) constraint, a medial (MED) range, and/or a bone constraint. The AP constraint may include a translation constraint and/or a coronal angulation constraint. The user may select the translation constraint as lateral or medial. Additionally, or alternatively, the user may input a range associated with the translation constraint.

The AP constraint may include a coronal angulation constraint. Similarly, the coronal angulation constraint may include a user selectable input of valgus or varus. Additionally, or alternatively, the user may input a range associated with the coronal angulation constraint. For example, the user may enter the range associated with the coronal angulation constraint as a numerical degree value. The range may correspond to acceptable limits for the constraint (e.g., the coronal angulation constraint). For example, the user may enter the range to correspond to an acceptable minimum value and/or an acceptable maximum value.

The LAT constraint may similarly include a user selectable input of anterior or posterior for a LAT translation constraint and/or a sagittal angulation apex constraint. The LAT translation constraint may include a user selectable range (e.g., in mm) associated with the input LAT translation constraint and/or a user selectable range (e.g., in degrees) associated with the input LAT sagittal angulation apex.

The user interface 310 may include a bone length constraint and/or a clinical rational deformity constraint. For example, the bone length constraint may include a user selectable input of too short or too long. Additionally, or alternatively, the user interface 310 may include a user selectable range (e.g., in mm) associated with the bone length constraint. Similarly, the clinical rational deformity constraint may include a user selectable input of internal or external. Additionally, or alternatively, the user interface 310 may include a user selectable range (e.g., in mm) associated with the clinical rational deformity.

FIG. 3C is a view of another example interface 320 for selection of ring constraints. The user interface 320 may include user selectable constraints associated with a proximal ring (e.g., first ring 110) and/or a distal ring (e.g., second ring 120). The constraints associated with the proximal ring (e.g., first ring 110) may include a ring type, a diameter, and/or a strut mount location. The user may select the ring type, for example from a drop down menu. Example ring types may include full, half, two-thirds, U-shaped, and/or oblong.

Additionally, or alternatively, the user may select a proximal ring diameter, for example from a drop down menu. In some examples, the user may enter any numerical value using a keypad (e.g., of the user interface). The user may enter a strut mount location, which may correspond to where a strut of the hexapod is to be mounted on the ring (e.g., first ring 110). For example, the user may enter tab mount or ring mount. Tab mount may refer to a strut being mounted on an aperture (e.g., aperture 114) on a tab (e.g., the protrusion portion 116) of the ring (e.g., first ring 110). Ring mount may refer to a strut being mounted on an aperture (e.g., aperture 112 on a first ring first apertures 112 adjacent an inner surface 115 of the first ring 110). In some examples, the user may select a specific aperture for mounting a (e.g., specific) strut. Similarly, the user interface 320 may include a selectable ring type, diameter, and/or strut mounting location associated with the distal ring (e.g., second ring 120).

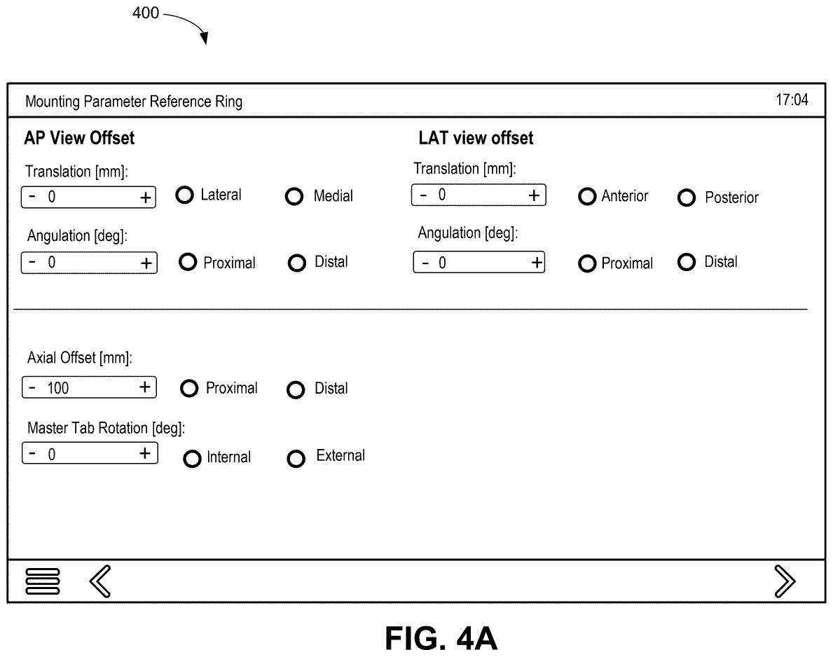

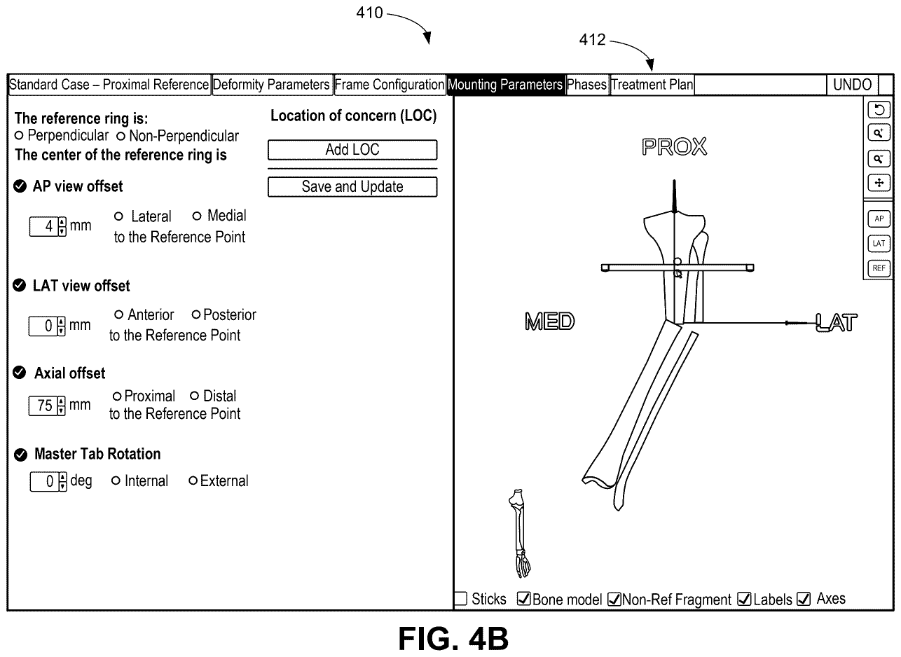

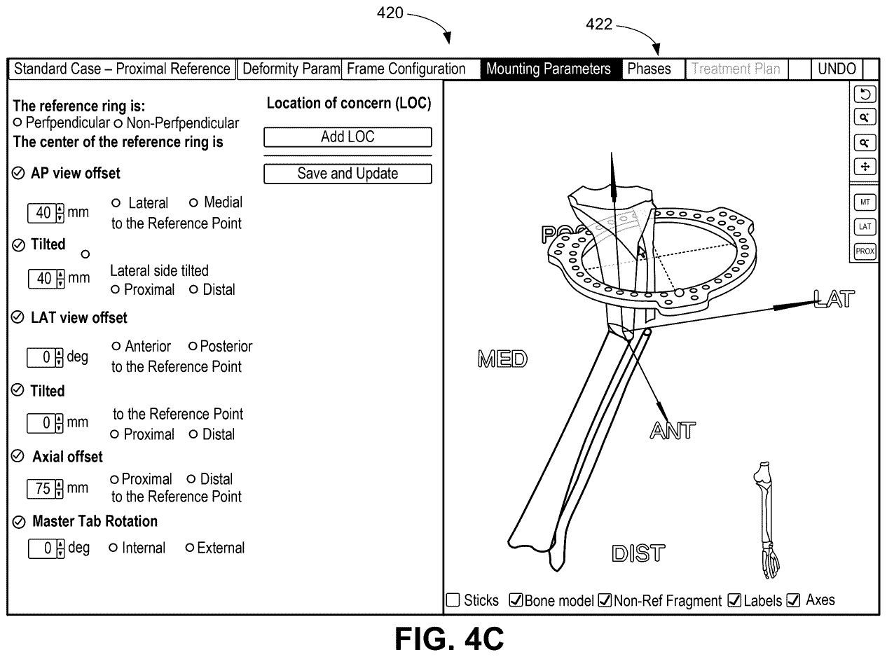

FIG. 4A is a view of an example interface 400 for selection of ring mounting constraints. The user may enter the body side (e.g., left or right) where the deformity is located. The user interface 400 may include an AP view offset constraint. The AP view offset constraint may be associated with a translation constraint and/or a coronal angulation constraint. As discussed herein, the AP translation constraint may include user selectable inputs including valgus, varus, and/or a range associated with coronal angulation constraint.

The user interface 400 may include a LAT view offset constraint. The LAT view offset constraint may be associated with a LAT view translation constraint and/or an angulation constraint. As discussed herein, the constraint (e.g., each constraint) may have an associated range. The user interface 400 may include a user selectable axial offset constraint. The axial offset constraint may include a user selectable input of proximal or distal. Additionally, or alternatively, the user may select a range associated with the axial offset constraint. The user interface 400 may include a user selectable master tab rotation constraint. The master tab rotation constraint may include a user selectable input of internal or external. Additionally, or alternatively, the user may select a range associated with the master tab rotation constraint.

FIG. 4B is a view of another example interface 410 for selection of ring mounting constraints. The user interface 410 may include a user selectable input of perpendicular or non-perpendicular, for example with respect to a position of the reference ring. As discussed herein, the user interface 410 may include an AP view offset constraint, a LAT view offset constraint, an axial offset constraint, and/or a master tab rotation constraint.

Additionally, or alternatively, the user interface 410 may include a graphical display 412 of a patient, for example a bone fragment of the patient. The user selection of the constraint may be visible in the graphical display 412. For example, the AP view offset constraint, the LAT view offset constraint, the axial offset constraint, and/or the master tab rotation constraint may be graphically displayed. User selectable inputs may be displayed, for example on the graphical display 412. For example, the user selectable inputs may be selected or deselected by a user. The user selectable inputs may include sticks, bone model, non-ref fragment, labels, and/or axes.

The graphical display 412 may include rotational and/or translational tools. The user may utilize the rotational and/or translational tools to adjust the graphical display 412, for example such that a desired view is displayed. The user may add a location of concern (LOC), for example associated with the bone fragment of the patient. For example, the user interface 410 may include a selectable input for the user to enter the LOC.

FIG. 4C is a view of another example interface 420 for selection of ring mounting constraints. Similar to the user interface 410, user interface 420 may include a user selectable input of perpendicular or non-perpendicular, for example with respect to a position of the reference ring. As discussed herein, the user interface 420 may include an AP view offset constraint, a LAT view offset constraint, an axial offset constraint, and/or a master tab rotation constraint. The user interface 420 may include a tilted constraint. The tilted constraint may include a user selectable input of proximal or distal, for example with respect to lateral side tilt. Additionally, or alternatively, the tilted constraint may include a user selectable range input. The user interface 420 may include a (e.g., second) tilted constraint, which may include a user selectable input of proximal or distal, for example with respect to the reference point. The (e.g., second) tilted constraint may additionally, or alternatively, include a user selectable range input.

The user interface 420 may include a graphical display 422 of a patient, for example a bone fragment of the patient. Similar to the graphical display 412 of user interface 410, the user selection of the constraint may be visible in the graphical display 422 and/or user selectable inputs may be displayed on the graphical display 422. For example, the user selectable inputs may be selected or deselected by a user. The graphical display 42 may include a component of the external fixator system (e.g., hexapod). For example, the graphical display 422 may include a display of the reference ring. Additionally, or alternatively, the graphical display 422 may include a display of the distal ring and/or one or more struts.

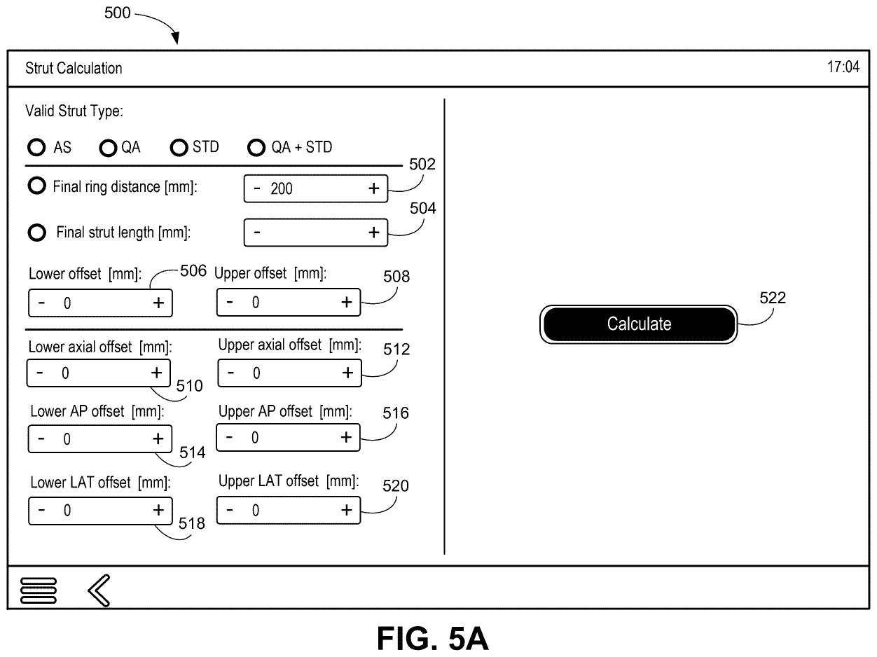

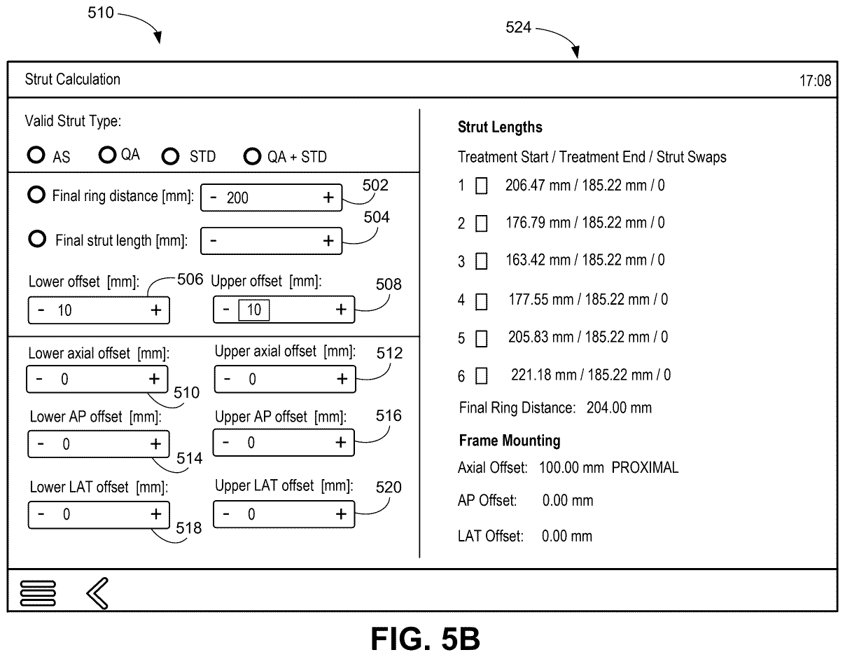

FIG. 5A is a view of an example interface 500 for calculating a hexapod strut configuration. The user interface 500 may include user selectable inputs for autostrut (AS), quick adjust (QA), standard (STD), and/or QA +STD. A user may input a value associated with a final ring distance constraint and/or a value associated with a final strut length constraint using the user interface 500. For example the user may input the value associated with the final ring distance constraint at 502 and/or the value associated with the final strut length constraint at 504. Additionally, or alternatively, the user may input one or more ranges associated with (e.g., each of) constraints on the user interface 500. Example constraints may include a lower offset constraint, an upper offset constraint, a lower axial offset constraint, an upper axial offset constraint, a lower AP offset constraint, an upper AP offset constraint, a lower LAT offset constraint, an upper LAT offset constraint, a rotation (e.g., rotation constraint), and/or a tilt (e.g., tilted constraint). The range (e.g., each range) may include a negative offset and/or a positive offset. The negative offset may indicate a range (e.g., an acceptable range) below a constraint value. For example, a negative offset of −3 mm may indicate a range of −3 mm below a set final ring distance constraint value of 200 mm. The positive offset may indicate a range (e.g., an acceptable range) above a constraint value. For example, a positive offset of 3 mm may indicate a range of 3 mm above a set final ring distance constraint value of 200 mm. In some examples, a user may set the offset (e.g., range) to a value for both the negative offset and the positive offset (e.g., 3 may indicate a negative offset of −3 and/or a positive offset of 3).

The lower offset 506 may be a range associated with the final ring distance constraint, for example at a lower position of the ring. Alternatively, or additionally, the lower offset 506 may be a range associated with the final strut length constraint at a lower portion (e.g., second end 134) of the strut. A user may enter the one or more ranges based on an acceptable range for the (e.g., each) respective constraint. Similarly, the upper offset 508 may be a range associated with the final ring distance constraint, for example at an upper position of the ring. Alternatively, or additionally, the upper offset 508 may be a range associated with the final strut length constraint at an upper portion (e.g., first end 132) of the strut. A range (e.g., each range) may correspond to a volume in which the component may be located. For example a range of 5 mm of the lower offset of the final strut length may correspond to the lower portion (e.g., second end 134) of the strut being located in a volume within the range of 5 mm.

Similarly the lower axial offset 510 may be a range associated with the final ring distance or final strut length axially from the bone (e.g., at a lower position). The upper axial offset 512 may be a range associated with the final ring distance or final strut length axially from the bone (e.g., at an upper position). The lower AP offset 514 may be a range associated with the final ring distance or final strut length anteroposterior from the bone (e.g., at a lower position). The upper AP offset 516 may be a range associated with the final ring distance or final strut length anteroposterior from the bone (e.g., at an upper position). The lower LAT offset 518 may be a range associated with the final ring distance or final strut length laterally from the bone (e.g., at a lower position). The upper axial offset 520 may be a range associated with the final ring distance or final strut length laterally from the bone (e.g., at an upper position).

The system may determine a mounting zone for the hexapod, for example including one or more strut mounting position(s) and/or ring location(s). The mounting zone may include one or more ranges. For example, the one or more ranges may include a range for each constraint. Example constraints may include an AP view offset constraint, a LAT view offset constraint, an axial offset constraint, and/or a master tab rotation constraint (e.g., as in FIG. 4C). Additionally, or alternatively, example constraints may include a tilt (e.g. tilted constraint) associated with the AP view offset and/or a tilt (e.g. tilted constraint) associated with the LAT view offset (e.g., as in FIG. 4C). The system may determine the subset of components based on the constraints (e.g., constraints as in FIG. 4C). The system may determine one or more outputs, for example the lower axial offset 510, the upper axial offset 512, the lower AP offset 514, the upper AP offset 516, the lower LAT offset 518, and/or the upper axial offset 520 (e.g., as in FIGS. 5A and 5B). The system may determine the mounting zone and/or the subset of components based on the outputs. For example, the system may determine the mounting zone and/or subset of components based on one or more ranges. The system may determine the subset of components based on the mounting zone. In some examples, the system may determine the mounting zone and/or the subset of components based on minimizing strut swaps during patient treatment with the external fixator system. For example, the system may determine one or more strut mounting position(s) and/or ring location(s) such that strut swaps are minimized. While example constraints are shown in FIG. 5, any constraints may be included for example with the user interface 500 and/or may be used to determine the subset of components. A (e.g., each) constraint may be associated with one or more ranges (e.g., offsets) as described herein.

The user interface may include a user selectable calculate button 522. The system may determine the subset of components, for example when the user selects the calculate button 522. The determination of the subset of components may be based on one or more of the constraints and/or the range of possible values for the constraint. Example constraints may include the upper ring location, a tolerance in the upper ring location, the lower ring location, a tolerance in the lower ring location, a strut mount location (e.g., on a ring and/or at an aperture), a strut length, a distance between the upper and lower rings (e.g., the first ring 110 and the second ring 120), an angle between the upper and lower rings (e.g., the first ring 110 and the second ring 120), an AP view offset, a LAT view offset, an axial offset, a master tab rotation, a rotation (e.g., rotation constraint), a tilt (e.g., tilted constraint), and/or a skin circumference. The strut length value may indicate a length of a strut, for example when configured between the upper ring mounting location and the lower ring mounting location. The range of values may indicate a tolerance in the lower ring mounting location, for example as a lower ring tolerance range. The upper range value may indicate a tolerance in the length of a strut, for example at an initial position of the strut, at an final position of the strut, and/or at a strut mount location (e.g., on a ring and/or at an aperture).

The system may determine the subset of components based on one or more of the constraints (e.g., entered by the user). For example, the system may determine the subset of components based on a plurality of the constraints. The system may weigh (e.g., different) constraints differently, such that one constraint is given more weight (e.g., weighted coefficient) than another constraint in the determination of the subset of components. For example, a user may assign a weight (e.g., weighted coefficient) to one or more of the constraints. The user may enter a weight for one or more constraints using the user interface. Additionally, or alternatively, the system may assign a weight (e.g., weighted coefficient) to one or more of the constraints, for example a predetermined and/or static weight. For example, the system and/or algorithm may have one or more predetermined weights (e.g., weighted coefficients) for one or more constraints.

FIG. 5B is a view of another example interface 510 for calculating a hexapod strut configuration. Similar to user interface 500, the user may input a range for (e.g., each) constraint. Once the system determines the subset of components, the user interface 510 may display an indication of the subset of components, for example at graphical display 524. The subset of components may include a plurality of strut sizes and/or ring sizes. For example, as shown in FIG. 5B the system may determine and/or display values associated with a strut. The values may include a length of the strut associated with treatment start and/or a length of the strut associated with treatment end. Additionally, or alternatively, the system may determine and/or display how many strut swaps will be expected during the treatment. For example, the system may determine and/or display the expected number of strut swaps for (e.g., each of) a particular strut of the hexapod.

The system may determine the subset of components based on different criteria, which may include one or more weights (e.g., weighted coefficient(s)). For example, the system may determine the subset of components based on minimizing strut swaps during patient treatment with the external fixator system. Additionally, or alternatively, the system may determine the subset of components based on minimizing weight (e.g., of the hexapod). For example, the system may determine the weight of (e.g., each of) the components (e.g., of the subset of components). The system may determine the subset of components based on a combination of minimizing weight and minimizing strut swaps. Additionally, or alternatively, the system may determine the subset of components based on frame (e.g., hexapod) stability. For example, a size of the hexapod (e.g., components) may be used by the system to determine the subset of components. Smaller components may result in greater frame (e.g., hexapod) stability and/or greater comfort for the patient.

The system may determine the subset of components based on minimizing strut swaps. Minimizing the strut swaps may be determined by the system by determining the minimum strut swaps for each strut (e.g., each of struts 1-6 in FIG. 5B). Additionally, or alternatively, minimizing strut swaps may be based on the length of the (e.g., each) strut and/or the range of the (e.g., each) strut. The system may determine the minimum number of strut swaps based on the mounting location of a (e.g., each) strut and/or the ring location(s). For example, the system may determine the minimum number of strut swaps based on the (e.g., determined) mounting zone. The system may determine and/or a user may enter the mounting position of a strut (e.g., on the upper ring and/or the lower ring) and/or the ring location(s). The mounting position may correspond to a specific aperture (e.g., on the ring). By determining the aperture to mount the strut, an effective length of the strut may change, such that the strut length constraint changes.

The system (e.g., algorithm) may determine options for a constraint value (e.g., final constraint value) and determine a component based on the options. For example, the system may determine that the range for the final strut length is 140-160 mm. The system may determine a strut (e.g., of a plurality of struts) based on the final strut length and/or other constraints. For example, the system may determine a strut such that a strut swap is not needed, based on the final strut length and/or other constraints. In some examples dimensions may be decoupled. For example, the system may decouple dimensions in the algorithm. The system (e.g., algorithm) may alternatively, or additionally, perform a Monte Carlo solution and/or a quick calculation.

The user may change one or more constraints, for example after the system has determined the subset of components. For example, the system may display an indication of the subset of components (e.g., after calculating the subset of components). The user may (e.g., then) change one or more of the constraints and the system may redetermine the subset of components (e.g., after the user actuates the calculate button). Additionally, or alternatively, the system may receive an additional plurality of constraints (e.g., from user input), for example with one or more changes to the plurality of constraints. The system may determine, based on the additional plurality of constraints, a subset (e.g., second subset) of components. The user may (e.g., then) choose one of the subsets of components.

The user interface 510 may display determined frame mounting constraints. For example, the frame mounting constraints may include an axial offset constraint, an AP offset constraint, and/or a LAT offset constraint. In some examples, the system may display one or more frame mounting constraints with entries determined by the user.

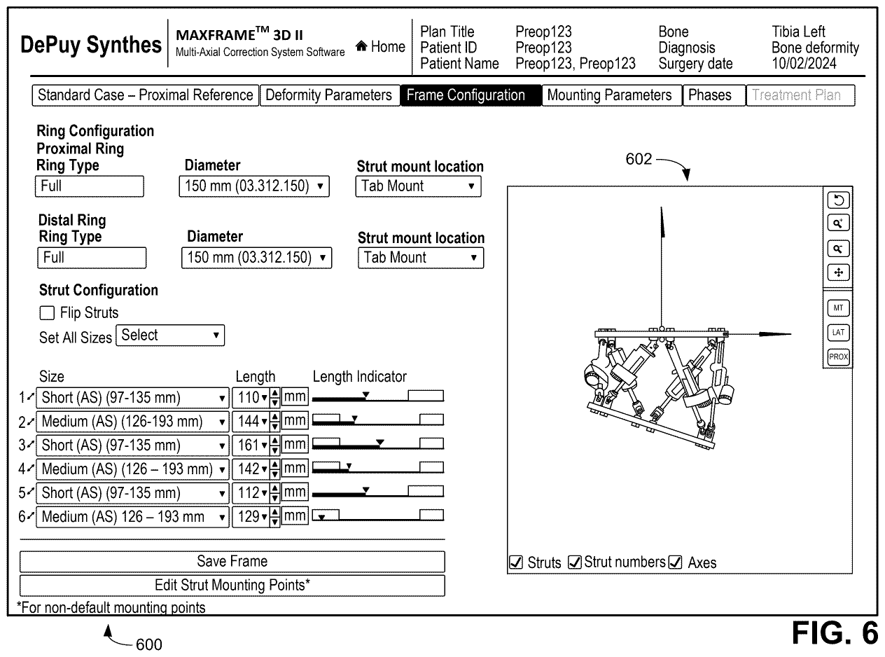

FIG. 6 is a view of an example interface 600 of a calculated output hexapod location. Similar to user interface 510, user interface 600 may display constraint outputs. Example constraint outputs may include a ring type, a ring diameter, and a strut mount location. The user interface may display constraint outputs for each of the proximal ring and for the distal ring. The user interface 600 may display strut configuration constraints. Example strut configuration constraints may include an indication of strut size, strut length, and/or a length indicator. The indication of strut size may include size term, for example short, medium, or long (e.g., for each strut). Additionally, or alternatively, the indication of strut size may include a numerical value associated with the strut size. The numerical value may include a range, for example indicating a minimum and maximum size of the strut. The length indicator may include a graphical representation of the strut length. For example the length indicator may include a bar graph.

The user interface 600 may include a graphical display 602, for example of the components of the hexapod. The determined subset of components may be visible in the graphical display 602. User selectable inputs may be displayed, for example on the graphical display. For example, the user selectable inputs may be selected or deselected by a user. The user selectable inputs may include struts, strut numbers, and/or axes. Selecting a selectable input may cause the user interface 600 to display the associated user input. Deselecting the selectable input may cause the user interface to remove the display of the associated user input.

The graphical display 602 may include rotational and/or translational tools. The user may utilize the rotational and/or translational tools to adjust the graphical display 602, for example such that a desired view is displayed. The user interface 600 may include an edit button. For example, the edit button may be for editing strut mounting points. In some examples the edit button may prompt the user to edit constraints. For example, the user may actuate the edit button to enter the one or more changes to the plurality of constraints. The system may redetermine the subset of components after the user edits one or more constraints.

What is claimed is:

1. At least one computer-readable medium for determining a subset of components of an external fixator system selected from an overall set of components of the external fixator system, the overall set of external fixator system including a plurality of struts of various sizes and a plurality of rings, the at least one computer-readable medium having stored thereon instructions that, when executed by at least one processor, cause the at least one processor to:
receive a plurality of constraints on the placement of components of the external fixator system relative to a bone, at least one of the plurality of constraints having a range of possible values for the constraint;
determine based on the received plurality of constraints, including the at least one of the plurality of constraints having a range of possible values for the constraint, the subset of components of the external fixator system comprising a selected plurality of strut sizes and ring sizes; and
display an indication of the subset of components.

2. The at least one computer-readable medium of claim 1, wherein the instructions, when executed by the at least one processor, causing the at least one processor to determine the subset of components based on the received plurality of constraints is further based on minimizing strut swaps during patient treatment with the external fixator system.

3. The at least one computer-readable medium of claim 1, wherein the subset of components comprises a first subset of components, wherein the instructions, when executed by the at least one processor, cause the at least one processor to determine a second subset of components, and to select the first subset of components or the second subset of components.

4. The at least one computer-readable medium of claim 3, wherein the plurality of constraints comprise a weight and wherein the instructions, when executed by the at least one processor, cause the at least one processor to determine the first subset of components based on the weight, and to determine the second subset of components based on minimizing strut swaps during patient treatment with the external fixator system.

5. The at least one computer-readable medium of claim 1, wherein the plurality of constraints comprise a first plurality of constraints and the subset of components comprises a first subset of components, and wherein the instructions, when executed by the at least one processor, cause the at least one processor to:
receive a second plurality of constraints on the placement of components of the external fixator system relative to a bone, at least one of the second plurality of constraints having a range of possible values for the constraint;
determine based on the received second plurality of constraints, including the at least one of the plurality of constraints having a range of possible values for the constraint, a second subset of components of the external fixator system comprising a second selected plurality of strut sizes and ring sizes; and
display an indication of the second subset of component.

6. The at least one computer-readable medium of claim 1, wherein the instructions, when executed by the at least one processor, cause the at least one processor to display an indication of a range associated with a ring, the range comprising a volume within which the ring may be located.

7. The at least one computer-readable medium of claim 1, wherein the instructions, when executed by the at least one processor, cause the at least one processor to display an arrow indicating a direction of a strut during patient treatment with the external fixator system.

8. The at least one computer-readable medium of claim 1, wherein the plurality of constraints comprises a skin circumference, and wherein the instructions, when executed by the at least one processor, cause the at least one processor to:
receive a value associated with the skin circumference; and
determine a ring comprising an inner circumference greater than or equal to the value associated with the skin circumference plus a predetermined offset.

9. The at least one computer-readable medium of claim 1, wherein the plurality of constraints comprises an upper ring location and the range of possible values for the constraint comprises a tolerance in the upper ring location, and wherein the instructions, when executed, further cause the at least one processor to determine, based on the tolerance in the upper ring location, whether to include a strut size in the in the subset of components.

10. The at least one computer-readable medium of claim 1, wherein the plurality of rings comprises an upper ring and a lower ring, the plurality of constraints comprises a first upper ring location and a second upper ring location opposite the first upper ring location, the plurality of constraints further comprises a first lower ring location and a second lower ring location opposite the first lower ring location, and wherein the instructions, when executed, further cause the at least one processor to:
determine a first distance between the first upper ring location and the first lower ring location;
determine a second distance between the second upper ring location and the second lower ring location; and
determine an upper ring mounting location and a lower ring mounting location based on the first distance and the second distance.

11. The at least one computer-readable medium of claim 1, wherein the instructions, when executed, further cause the at least one processor to:
determine a weight associated with each respective constraint of the plurality of constraints; and
wherein the instructions, when executed by the at least one processor, causing the at least one processor to determine the subset of components based on the received plurality of constraints is further based on the weight associated with each respective constraint of the plurality of constraints.

12. The at least one computer-readable medium of claim 1, wherein the indication of the subset of components comprises a respective color indicator corresponding to each strut size of the selected plurality of strut sizes.

13. The at least one computer-readable medium of claim 1, wherein the external fixator system comprises a hexapod.

14. A method for determining a subset of components of an external fixator system selected from an overall set of components of the external fixator system, the overall set of external fixator system including a plurality of struts of various sizes and a plurality of rings, the method comprising:
receiving a plurality of constraints on the placement of components of the external fixator system relative to a bone, at least one of the plurality of constraints having a range of possible values for the constraint;
determining based on the received plurality of constraints, including the at least one of the plurality of constraints having a range of possible values for the constraint, the subset of components of the external fixator system comprising a selected plurality of strut sizes and ring sizes; and
displaying an indication of the subset of components.

15. The method of claim 14, wherein determining the subset of components based on the received plurality of constraints is further based on minimizing strut swaps during patient treatment with the external fixator system.

16. The method of claim 14, wherein the subset of components comprises a first subset of components, the method further comprising:
determining a second subset of components; and
selecting the first subset of components or the second subset of components.

17. The method of claim 16, wherein the plurality of constraints comprise a weight, the method further comprising:
determining the first subset of components based on the weight; and
determining the second subset of components based on minimizing strut swaps during patient treatment with the external fixator system.

18. The method of claim 14, wherein the plurality of constraints comprise a first plurality of constraints and the subset of components comprises a first subset of components, the method further comprising:
receiving a second plurality of constraints on the placement of components of the external fixator system relative to a bone, at least one of the second plurality of constraints having a range of possible values for the constraint;
determining based on the received second plurality of constraints, including the at least one of the plurality of constraints having a range of possible values for the constraint, a second subset of components of the external fixator system comprising a second selected plurality of strut sizes and ring sizes; and
displaying an indication of the second subset of component.

19. The method of claim 14, further comprising displaying an indication of a range associated with a ring, the range comprising a volume within which the ring may be located.

20. At least one computer-readable medium for determining a subset of struts of a plurality of struts, the at least one computer-readable medium having stored thereon instructions that, when executed by at least one processor, cause the at least one processor to:
determine a strut length value, a lower range, and an upper range, wherein the strut length value indicates a length of a strut when configured between an upper ring mounting location and a lower ring mounting location;
determine the subset of hexapod struts of the plurality of hexapod struts based on the strut length value, the lower range, and the upper range, wherein the plurality of hexapod struts are associated with different ranges of lengths; and
display an indication of the determined subset of struts.
